(19) United States
(12) Patent Application Publication
(10) Pub. No.: US 2010/0185131 A1
(43) Pub. Date: Jul. 22, 2010

(54) DEVICE FOR CORRECTING BOWLEG

(76) Inventor: Kyung-Bae Seo, Seoul (KR)

Correspondence Address: LERNER, DAVID, LITTENBERG,;KRUMHOLZ & MENTLIK, 600 SOUTH AVENUE WEST, WESTFIELD, NJ, 07090, US

(21) Appl. No.: 12/440,048

(22) PCT Filed: Apr. 16, 2007
(86) PCT No.: PCT/KR07/01828

§ 371 Date: Mar. 5, 2009

(30) Foreign Application Priority Data
Sep. 12, 2006 (KR) 10-2006-0088157

(51) Int. Cl. A61F 5/00

(52) U.S. Cl. 602/32

(57) ABSTRACT

A device for correcting bowleg includes a pair of knee holders for rotating together with knees while holding the knees, a rotational driving unit for repeatedly driving the knee holders forward and backward in directions of rotating the knees outward and inward, respectively, and a controller having a memory storing rotation conditions of the knee holders. The controller controls the rotational driving unit according to the rotation conditions stored in the memory. A device for correcting bowleg includes a pair of foot supports for receiving and supporting feet, the foot supports capable of performing forward and backward rotations in directions of rotating the feet outward and inward, respectively, and a third resisting means for resisting against the forward rotation of the foot supports.

### TECHNICAL FIELD

The present invention relates to a device for correcting bowleg, particularly, which can be used for different body structures of various users to provide optimal correction effects to the users without applying excessive load to the bodies of the users.

### BACKGROUND ART

The hip joint is an articulation between the pelvis and the femur, located inside a bone protruding from the side of the hip. The inward rotation deformity in the hip joint (bowleg deformity) causes the legs to bow in the shape of ‘O ’. The bowleg has been corrected by surgery. However, such surgery takes a large amount of money and time due to complicated procedures, and furthermore is dangerous.

Therefore, the applicant has developed a device capable of simply and effectively correcting bowleg by forcing the knees to rotate outward. In the course of development, a test version of the correction device was also proposed.

The test version of correction device, however, is merely focused on forcing the knee to rotate outward, and thus has the following problems.

First, the test version of correction device cannot properly cope with different body structures of users such as different skeletal structures and different cartilage tissues, different slopes of knee joint surfaces, and different levels of inward rotation deformities, and thus applies excessive load to user bodies. In some cases, it is even impossible to use the correction device for this reason.

This kind of correction device is directly applied to human bodies as a medical device or a medical aid. Therefore, even if the correction device has high correction effects, it is improper to use this device as the medical device or the medical aid if there is a possibility of damaging the human bodies.

Second, the test version of correction device shows very poor power efficiency. When the knees are forced to rotate in the state where they are not firmly fixed, knee supports rotate idle without rotating the knees. Since it is structurally difficult for the test version device to firmly fix the knees, the knee supports slip on the knees and thus the device has extremely poor efficiency. In practice, when various test version devices were tested, the knees rarely rotated due to the slip.

Third, the test version of correction device cannot reflect the exercise history of the user, and thus cannot obtain optimal correction effects.

Fourth, the test version of correction device can only obtain correction effects from the rotation of the knees, but not from the rotation of feet, and thus correction effects are limited.

### DISCLOSURE OF INVENTION

### Technical Problem

The present invention has been made to solve the foregoing problems with the prior art and therefore an object of the present invention is to provide a correction device which can maximize correction effects irrespective of different body structures of users without applying excessive load to the bodies of the users.

Another object of the present invention is to provide a correction device in which the rotation of knee supports leads to the rotation of knees without loss, to minimize the slip of knee supports and obtain excellent power efficiency.

A further object of the present invention is to provide a correction device, which can reflect the exercise history of users in order to provide optimal correction effects. Since the upper limit of an exercise stage which the user can select, is adjusted according to the exercise history of the user, it is possible to ensure optimal correction effects without applying excessive load to the user.

Yet another object of the present invention is to provide a correction device which can obtain correction effects through the rotation of feet together with or separately from correction effects through the rotation of knees.

### Technical Solution

According to an aspect of the invention for realizing the object, the device for correcting bowleg includes a pair of knee holders for rotating together with knees while holding the knees, a rotational driving unit for repeatedly driving the knee holders forward and backward in directions of rotating the knees outward and inward, respectively, and a controller having a memory storing rotation conditions of the knee holders, wherein the controller controls the rotational driving unit according to the rotation conditions stored in the memory.

Preferably, the knee holders are laterally freely-movable within a first preset range.

Preferably, the knee holders are horizontally freely-rotatable within a second preset range.

Preferably, each of the knee holders includes a knee protector coupled to the knee and a knee support for supporting the knee. The knee support is driven by the rotational driving unit, and the knee protector rotates along with the knee support, thereby rotating the knee. In addition, the knee protector is relatively freely-movable with respect to the knee support within a third preset range.

Preferably, the device further includes a pair of foot supports, wherein the foot supports receive and support feet while the knee holders hold the knees, the foot supports being capable of performing forward and backward rotations in directions of rotating the feet outward and inward, respectively.

Preferably, the memory stores an exercise history of a user, and the controller searches for a rotation condition corresponding to the exercise history of the user stored in the memory, and controls the rotational driving unit according to the searched rotation condition.

According to another aspect of the invention for realizing the object, the device for correcting bowleg includes a pair of foot supports for receiving and supporting feet, the foot supports being capable of performing forward and backward rotations in directions of rotating the feet outward and inward, respectively, and means for resisting against the forward rotation of the foot supports.

### ADVANTAGEOUS EFFECTS

As set forth above, the correction device of the present invention has first to third preset ranges allowing the knee holders to freely-move or freely-rotate and thus can maximize correction effects irrespective of different body structures of users without applying excessive load to the bodies of the users.

Also, the correction device of the present invention can properly transmit the rotation of the knee supports to the rotation of the knees to minimize the slip of knee supports and obtain excellent power efficiency.

Furthermore, the correction device of the present invention can reflect the exercise history of users to provide optimal correction effects.

Moreover, the correction device of the present invention can obtain correction effects through the rotation of feet together with or separately from correction effects through the rotation of knees.

### BRIEF DESCRIPTION OF THE DRAWINGS

FIG. 1 is a perspective view illustrating the external appearance of a correction device according to an exemplary embodiment of the present invention;

FIG. 2 is an exploded perspective view of the correction device shown in FIG. 1, from which the cover is removed;

FIG. 3 is an exploded perspective view of an assembly for a right knee shown in FIG. 2;

FIG. 4 is a top perspective view of the right supporter shown in FIG. 3;

FIG. 5 is a bottom perspective view of a right knee protector;

FIG. 6 is a front perspective view of an assembly for a foot shown in FIG. 2, from which the front cover is removed;

FIG. 7 is a rear perspective view of the assembly for a foot shown in FIG. 2, from which the rear cover is removed;

FIG. 8 is a process flowchart of the correction device shown in FIG. 1;

FIGS. 9 to 13 are diagrams sequentially illustrating the operation of the knee holder of the correction device shown in FIG. 1;

FIG. 14 is a graph illustrating the angle of exercise of the knee support according to the number of rotations in one set;

FIG. 15 is a perspective view schematically illustrating the external appearance of a correction device according to another embodiment of the present invention;

FIG. 16 is a perspective view illustrating the internal structure of the correction device shown in FIG. 15; and

FIG. 17 is a bottom perspective view of the switch locker shown in FIG. 16.

### BEST MODE FOR CARRYING OUT THE INVENTION

The present invention will now be described more fully with reference to the accompanying drawings, in which exemplary embodiments thereof are shown.

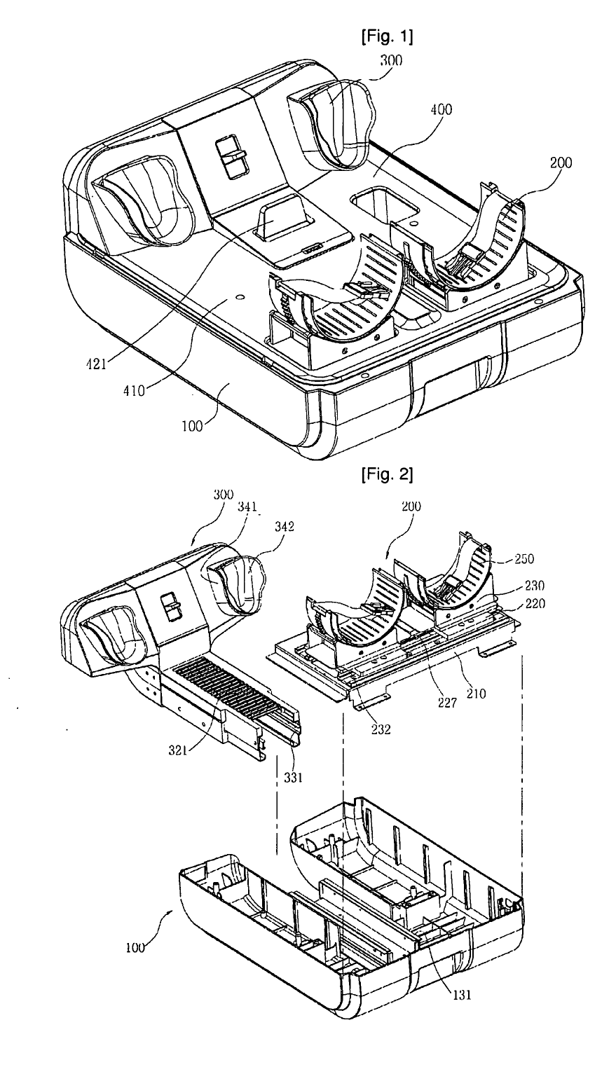

FIG. 1 is a perspective view illustrating the external appearance of a correction device according to an exemplary embodiment of the present invention, and FIG. 2 is an exploded perspective view of the correction device shown in FIG. 1, from which a cover 400 is removed. (A knee protector 271 shown in FIG. 5 is not depicted in FIGS. 1 to 3.)

The correction device includes a housing 100, an assembly 200 for a knee, an assembly 300 for a foot and a controller (not shown). The assembly 200 for a knee is fixedly attached to the housing 100, and the assembly 300 for a foot is movably attached to the housing 100 in such a manner that the assembly 300 can move back and forth.

The housing 100 has a housing cover 400, which has a cover plate 410 and a fixing unit. The fixing unit is fixedly attached to the cover plate 410. The fixing unit includes a lever 421, a wedge (not shown) and a spring (not shown). The wedge is engaged with a toothed part 321 in the assembly 300 by means of the force of the spring, thereby fixing the assembly 300. When the user pulls the lever 421 toward him/her, the wedge is lifted up against the force of the spring, thereby disengaged from the toothed part 321 of the assembly 300. Then, the user can move the assembly 300 back and forth. The fixing unit and the toothed part 321 function as a distance-adjusting means for adjusting the distance between knee holders 250 and foot supports 341.

Since the distance between the knee holders 250 and the foot supports 341 is adjustable, any user can use the device irrespective of his/her height.

The assembly 200 for a knee includes a base 210, seats 220, supporters 230, rotational driving units 240 and the knee holders 250. The base 210 is fixedly attached to the housing 100. The knee holder 250 is a component that rotates the knee while holding it. The rotational driving unit 240 rotates the knee holder 250 forward or backward in the direction of rotating the knee outward or inward, respectively.

The assembly 300 has a rail 331, which is movable back and forth along a rail 131 of the housing 100. Therefore, the assembly 300 moves back and forth with respect to the housing 100 in response to the relative movement between the rail 331 and the rail 131.

The controller has a memory storing rotation conditions (e.g., a rotation angle) of the knee holder, and controls the rotational driving unit according to the rotation conditions stored in the memory.

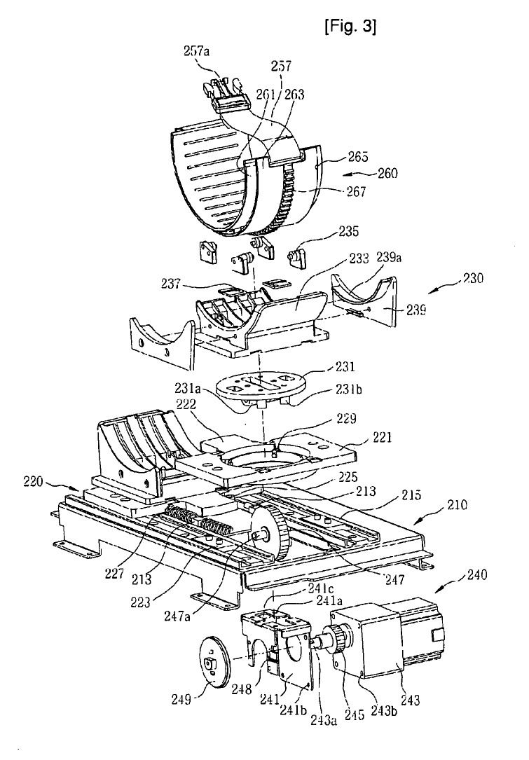

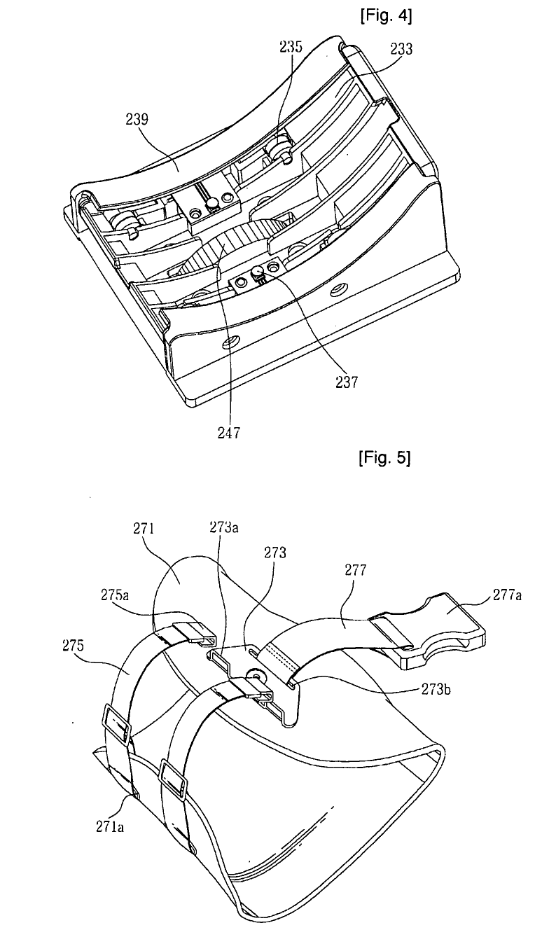

FIG. 3 is an exploded perspective view of the assembly 200 for a right knee shown in FIG. 2, and FIG. 4 is a top perspective view of the right supporter 230 shown in FIG. 3. (In FIG. 3 and/or FIG. 4, etc. is not shown.)

The base 210 includes hangers 213 and rails 215.

The seats 220 placed on the base 210 are freely-movable in the lateral direction within a preset range. Each of the seats 220 includes a plate 221, a protrusion 222, a slider 223, a link 225, first resisting means 227, and rollers 229.

This structure makes the knee holder 250 be freely-movable laterally within the first preset range. Accordingly, users can use the correction device of the present invention without suffering excessive load on their bodies.

Also, the user can enhance the correction effects by making his/her knees to move toward each other against the resistance of the resisting means 227. The first resisting means 227 resist against the plates 221 moving toward each other.

Although not shown in the drawings, it is preferable that the resisting force of the first resisting means 227 is adjustable. The first resisting means with the adjustable resisting force can be obtained, for example, by using a structure shown in FIG. 7 or an air spring.

The slider 223 is fixedly attached to the bottom of the plate 221. Since the slider 223 is movable to the right or left along the rail 215 of the base 210, the plate 221 is also movable to the right or left.

The protrusions 222 protrude from confronting parts of the plates 221, and enable the plates 221 to maintain a minimum distance therebetween.

The links 225 also protrude from confronting parts of the plates 221. The first resisting means 227, such as a spring, are combined between the link 225 of the seat 220 and the hanger 213 of the base 210.

The supporter 230 is mounted on the seat 220, and the rollers 229 of the plate 221 allow the supporter 230 to horizontally freely-rotate within a preset range.

The supporter 230 includes a bottom plate 231, a mount 233, rollers 235, a first sensor 237 and cover blocks 239.

The bottom plate 231 is a component that is seated on the rollers 229 of the seat 220. The bottom plate 231 can horizontally freely-rotate within a second preset range.

This makes the knee holder 250 mounted above the bottom plate 231 freely-rotate horizontally within the second preset range.

Since the knee holder 250 is designed to have the second preset range, users can use the correction device without suffering excessive load on their knee joint irrespective of the angle thereof.

That is, since the slopes of knee joint surfaces are different for different people, the knee joint surfaces can define right angle planes with respect to the height direction or be inclined with respect to the right angle planes. Accordingly, the knee supports 260 is designed to freely-rotate horizontally within the second preset range in consideration of the different slopes of the knee joint surfaces of users in order not to apply excessive load to the their knee joints.

The second preset range enables the knee holder 250 to securely and stably hold the knee. If the second preset range is not provided and thus, the centerline of the knee holder 250 is misaligned with that of the knee, the knee holder 250 can rarely hold the knee firmly.

The bottom plate 231 has a bearing 231a and fixing parts 231b on the underside thereof.

The mount 233 is fixedly attached to the top of the bottom plate 231, and the rollers 235 and the first sensor 237 are fixedly attached to the mount 233. The cover blocks 239 are fixedly attached to front and rear parts of the mount 233.

The cover blocks 239 act as covers to prevent the knee holder 250 from separating from the supporter 230, and have a protruded rim 239a.

The knee holder 250 includes a knee support 260, a knee protector 271 and a connector.

The knee support 260 seats and supports the knee, and is supported by the rollers 235 of the supporter 230, so that it can rotate forward and backward.

The knee support 260 includes two arcuate rims 261 and 263, located in the front and the rear. The rollers 235 of the supporter 230 are in touch with the outer surface of the arcuate rim 263. The protruding rim 239a of the cover block 239 is located in a space between the arcuate rims 261 and 263 of the knee support 260, thereby preventing the knee support 260 from being separated. That is, when the knee support 260 is forced upward, the inner surface of the outer arcuate rim comes into contact with the knee support 260, thereby preventing the knee support 260 from being separated.

In the case of the right knee support 260, an indicator 265 is provided in the right and rear end of the arcuate rim 263. Although not shown, another indicator is provided in the left and front end of the arcuate rim 263. A magnet can be used as the indicator 265. The first sensor 237 detects the indicator 265 to locate the position of the knee supports 260.

The first sensor 237 and the indicator 265 function as a first sensor mechanism that locates the position of the knee holder 250. The first sensor mechanism performs two functions: The first function of the sensor mechanism is to detect whether or not the knee holder 250 reaches a limit position beyond the normal rotation range. Then, the rotational driving unit 240 immediately stops the rotation of the knee holder 250. Since the knee rotated beyond the limit position leads to a medical accident, the first sensor mechanism is provided to prevent such a danger.

The second function of the sensor mechanism is to detect a reference position. The sensor mechanism identifies the limited position of the knee holder 250 as the reference position. More particularly, when driven by a predetermined amount of driving force, the knee holder 250 rotates from the reference position to a specific position which is identified as a starting position. Then, the user can use the correction device. Therefore, a separate sensor mechanism for detecting the starting position is not required. The first sensor mechanism used for detecting the limited position can be used to detect the starting position, which provides advantages such as a simplified structure and a lowered cost.

Gear teeth 267 are formed along the outer circumference of the knee support 260.

The rotational driving unit 240 is arranged under the bottom plate 231.

The rotational driving unit 240 includes a bracket 241, a driving motor 243, a driving gear 245, an intermediate gear 247, a second sensor 248, and a disk 249.

The bracket 241 is fixed to the bottom plate 231. The bracket 241 has screw holes 241a, through which bolts are fastened to the fixing parts 231b of the bottom plate 231, thereby fixing the bracket 241 to the underside of the bottom plate 231. Accordingly, the bracket 241, that is, the rotational driving unit 240 can horizontally freely-rotate within the second, preset range together with the supporter 230.

The driving motor 243 is fixed to the bracket 241. Bolts are fastened to screw holes 243b of the driving motor 243 and the screw holes 241b of the bracket 241, thereby fixing the driving motor 243 to the bracket 241.

A shaft 247a is inserted into the bearing 231a of the bottom plate 231 to support the intermediate gear 247. The body of the intermediate gear 247 is located to pass through a slot 241c of the bracket 241.

The driving force of the driving motor 243 is transmitted through the driving gear 245 and the intermediate gear 247 to the gear teeth 267 on the outer circumference of the knee support 260, thereby rotating the knee support 260.

The disk 249 is fixed to the end of a shaft 243a of the driving motor 243. A plurality of magnets is provided to the disk 249, along the circumferential direction of the disk 249.

The second sensor 248 is fixed to the bracket 241, and detects the magnets embedded in the disk 249. The second sensor 248 and the magnets embedded in the disk 249 constitute a second sensor mechanism. The second sensor mechanism detects the amount of rotation driven by the rotational driving unit 240. The detected amount of rotation is used for feedback control.

If the output amount of rotation detected by the second sensor mechanism is different from the input amount of rotation inputted to the rotational driving unit 240, and if the difference is beyond an error range, it is identified as an error, and thus the rotation of the knee holder 250 is stopped.

The second sensor mechanism, provided in addition to the first sensor mechanism, inspects whether or not the correction device is properly operating according to settings, thereby further enhancing the safety thereof. That is, the malfunctions of the correction device are inspected by both the first and second sensor mechanisms to prevent accidents. This enables the knee support 260 to rotate according to a rotation program which is set to provide optimal clinical treatment effects, and thereby the user can obtain optimal treatment effects.

FIG. 5 is a bottom perspective view of the knee protector 271 of the right knee holder 250.

The knee holder 250 has the knee support 260 for seating and supporting the knee, the knee protector 271 coupled to the knee, and the connector for connecting the knee protector 271 to the knee support 260. The knee support 260 is rotated by the rotational driving unit 240, and the knee protector 271 rotates along with the knee support 260 to rotate the knee.

The knee protector 271 has a shape conforming to the knee, and fixedly surrounds the knee. The knee protector 271 includes inner and outer plies, in which the inner ply contacting the knee is made softer than the outer ply.

The soft inner ply provides two merits: The first merit is to protect the knee from scratches. The second merit is to prevent the knee protector from slipping on the knee. As mentioned above, in the correction device of the present invention, preventing the slip between the knee and the knee protector 271 is very important. Thus, the merit obtained from the soft inner ply is also important.

However, if the whole part of the knee protector 271 is made of a soft material, the knee protector 271 cannot execute its own function of forcing the knee to rotate. Hence, the outer ply is made of a high-hardness material so that it can act as a structural component.

The connector is provided between the knee support 260 and the knee protector 271, and transmits forward rotation of the knee support 260 to the knee protector 271, thereby rotating the knee protector 271 in a forward direction.

The connector can be a belt which is fixed to the knee support 260 at one end thereof and extends in the direction of backward rotation at the other end thereof. In the shown embodiment, the connector includes a connector belt 257 attached to the knee support 260 and a connector belt 277 attached to the knee protector 271.

A fixing part 273 is fixed to the knee protector 271, and includes rings 273a and a slit 273b. Hooks 275a are provided at one end of fixing belts 275, and are hooked into the ring 273a. One end of the connector belt 277 is inserted into and fixedly fastened to the slit 273b.

The other ends of the fixing belts 275 are inserted into and fixedly fastened to slits 271a of the knee protector 271. The fixing belts 275 have an adjustable length.

The fixing belts 275 can have various forms. For example, the fixing belt can be designed to surround the knee protector 271 in such a fashion that one end of the fixing belt is fastened with the other end thereof. In this case, Velcro members can be provided to both ends of the fixing belt so that the both ends can be fastened with each other.

A buckle 277a is provided in the other end of the connector belt 277. The buckle 277a of the connector belt 277 is fastened with a buckle 257a of the connector belt 257. Thus, when the knee support 260 rotates forward, the connector belt 257 and the connector belt 277 cooperate to pull the knee protector 271 in a forward direction, thereby rotating the knee protector 271 in a forward direction.

However, when the knee support 260 rotates backward, it is impossible to push the knee protector 271. Hence, the knee support 260 rotates backward independent of the knee protector 271, and the knee also returns via inward rotation by itself. Here, the return angle of the knee is restricted by the return angle of the knee support 260.

The structure of connecting the knee protector 271 with the knee support 260 as mentioned above is one of the key features of the present invention.

According to the test version of correction device of the applicant, the knee is merely tied to the knee support in order to fix the knee. However, it is difficult to firmly tie the knee of the human body to the knee support. It is not easy to firmly tie the knee to the knee support by manpower only, and even if the knee is very firmly tied to the knee support, this causes pain in the knee. On the other hand, if the knee is loosely tied, the knee support 260 would slip on the knee.

Therefore, the structure for firmly fixing the knee to the knee support has been requested. The present invention solves this problem using a very simple structure based upon a new concept. Instead of fixing the knee to the knee support, the knee support 260 and the knee protector 271 are connected by the belts so that the knee support 260 can pull the knee to rotate together with the knee, when it is rotating forward.

The merit of this structure is also obtained in the backward rotation of the knee support 260. In the correction device of the present invention, the inward rotation of the knee is meaningful only as a return operation for the following forced outward rotation of the knee. It is not necessary to apply forced inward rotation to the knee. Rather, the forced inward rotation of the knee may not be preferable from the medical point of view.

Accordingly, in the present invention, the outward rotation of the knee is forced but the inward rotation thereof is made by itself.

According to another test version of correction device of the applicant, the knee protector 271 is fixed to the knee support 260 while surrounding the knee in such a manner that the knee protector 271 is never allowed to relatively freely-move with respect to the knee support so that the knee protector 271 can rotate forward or backward as the knee support 260 rotates forward or backward.

However, this structure may apply excessive load to the body of the user, since it can permit only one relative location between the knee (protector) and the knee support, and thus, it is not preferable to be used for various body structures of different users.

Accordingly, in the present invention, the knee support 260 and the knee protector 271 are connected together via the connector belts 257 and 277, so that the knee protector 271 can freely-move relatively with respect to the knee support 260 within a third preset range due to the twisting, bending, etc. of the connector belts 257 and 277.

However, it should not be construed that the scope of the present invention excludes the structure of simply tying and fixing the knee surrounded by the knee protector 271, to the knee support 260.

As described above, the knee holder 250 has the first to third ranges, which are attained through the independent structures. However, the present invention can have other various structures that afford the preset ranges. For example, an elastic member of rubber can be interposed between the seat and the supporter, so that the knee holder has the second and third ranges due to the intrinsic property of the elastic member. That is, the key concept of the present invention is to provide the first to third ranges to the knee holder 250 but the detailed structure for realizing this concept can be embodied in various forms.

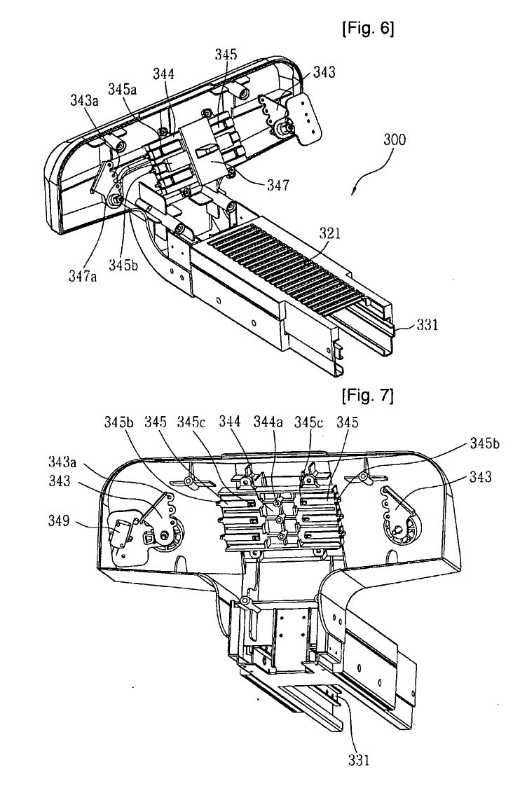

FIG. 6 is a front perspective view of the assembly 300 for a foot shown in FIG. 2, from which the front cover is removed. FIG. 7 is a rear perspective view of the assembly 300 shown in FIG. 2, from which the rear cover is removed.

The assembly 300 includes a pair of foot supports 341, a pair of rotation blocks 343, second resisting means (not shown), a substrate 344, another resisting means (not shown), link blocks 345, a switch locker 347 and third sensors 349.

In a state where the knee holder 250 holds the knee, that is, the knee support 260 stably supports the knee, the foot supports 341 receive and support the feet. The foot supports 341 are designed to be rotatable forward or backward in the direction of the outward or inward rotation of the feet.

Each foot support 341 has a footing cushion 342 which is composed of two plies: an inner ply contacting the foot, and an outer ply disposed in the outer side. The inner ply is made of a softer material than the outer ply which is preferably made of a high-hardness material. The footing cushion 342 prevents any pain that would otherwise be caused when the foot contacts the foot support 341.

The rotation blocks 343 are placed in the rear of respective foot supports 341. Each of the rotation blocks 343 is combined to the foot support 341 to rotate along with the foot support 341. The rotation block 343 has three rings 343a protruding toward respective link blocks 345.

The link blocks 345 are mounted on the substrate 344 to be horizontally movable to the right or left.

Each link block 345 has a groove 345a in the front side thereof.

Each link block 345 has a ring 345b protruding toward the rotation block 343. The second resisting means are fixed between the ring 343a of the rotation block 343 and the ring 345b of the link block 345. For example, coil springs with different spring constants can be used as the second resisting means.

A ring 345c is also provided on the rear side of the link block 345. Three fixing parts 344a are correspondingly provided on the rear side of the substrate 344, along the center line thereof. Fixing bolts (not shown) are screwed into the fixing parts 344a, resisting means (not shown) are provided between the fixing bolts and the rings 345c on the rear side of the link blocks 345. For example, a coil spring can be used as the resisting means.

The resisting means fixed to the ring 345c of the link block 345 may be a coil spring which has a spring constant smaller than that of the second resisting means fixed to the rings 345b of the link block 345 (a coil spring which is easily lengthened with a small amount of force).

The switch locker 347 is mounted to be vertically movable. The switch locker 347 has protruding parts 347a that protrude to the rear. Each of the protruding parts 347a is inserted into the groove 345a of one of the link blocks 345 to fix the same link block 345. Then, the other link blocks 345 are subject only to the resisting means with the smaller spring constant which are fixed to respective rings 345c, and thus can move relatively freely to the right or left.

Therefore, the link block 345 that is fixed by the switch locker 347 determines the magnitude of the resisting force against the rotation of the rotation block 343, that is, the forward rotation of the foot support 341. Because the second resisting means fixed to respective rings 345b of the link blocks 345 have different spring constants, the resisting force can be adjusted by moving the switch locker 347.

The resisting force against the outward rotation of the foot has to be adjusted to obtain optimal treatment effects for the body characteristics and conditions of the user.

The third sensor 349 has a switch at a position that can come in contact with the link block 345 when the link block 345 rotates forward at a predetermined angle or more. Hence, the third sensor 349 can detect the foot support 341 when it rotates at a predetermined angle or more.

The angle of the forward rotation of the foot support 341 to be detected by the third sensor 349 can be adjustably set. For example, the angle of the forward rotation of the foot support 341 can be adjusted through the relocation of the third sensor 349.

Because body structures and muscle strengths of respective users are different from each other, the present invention adjusts the degrees of the outward rotation of the foot differently according to the users in order to obtain optimal correction effects for respective users.

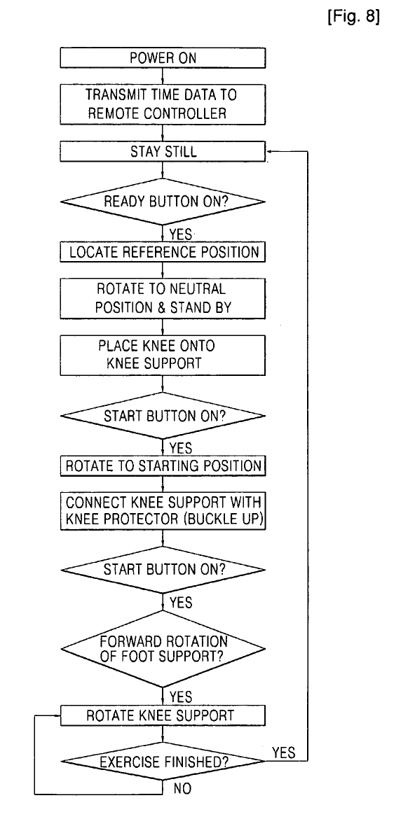

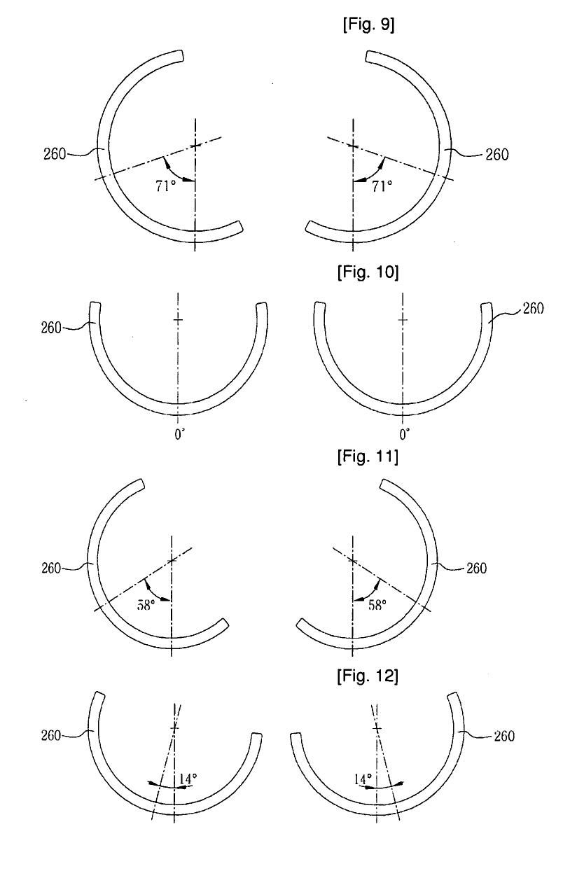

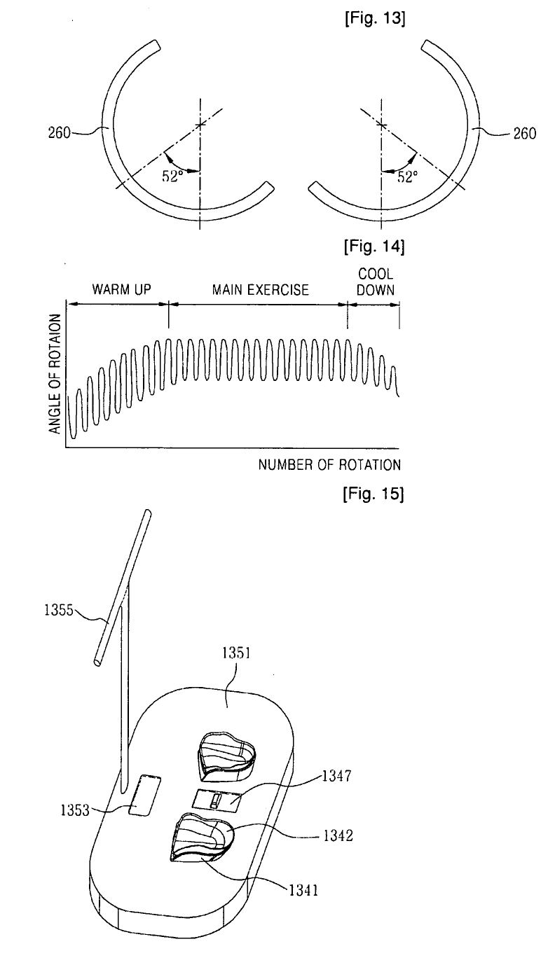

FIG. 8 is a process flowchart of the correction device shown in FIG. 1, FIG. 9 is a diagram illustrating the rotation angle of the knee support 260 at a reference position, FIG. 10 is a diagram illustrating the rotation angle of the knee support 260 at a neutral position, FIG. 11 is a diagram illustrating the rotation angle of the knee support 260 at a starting position, FIG. 12 is a diagram illustrating the rotation angle of the knee support 260 that has completed the first forward rotation, FIG. 13 is a diagram illustrating the rotation angle of the knee support 260 that has completed the first backward rotation, and FIG. 14 is a diagram illustrating the rotation angle of the knee support according to the number of rotations in one set.

When the correction device is powered on, it stays still.

Even in this state, it is possible to select exercise stages and the number of rotations.

When “READY” button on a remote controller is pressed, the knee supports 260 start rotating to detect a reference position.

The detection of the reference position is a procedure that the first sensor 237 detects the indicator 265. In the case of the right knee support 260 (hereinafter abbreviated as “knee support 260”), the indicator 265 at the front part of the outer circumference of the knee support 260 is placed at an angle of +71° (in the case of right knee support 260, “+” indicates clockwise direction) from the center of the arc of the knee support 260. The indicator 265 at the rear part of the outer circumference of the knee support 260 is placed at an angle of −83° from the center of the arc of the knee support 260.

The front indicator 265 is used to detect the reference position. When the front indicator 265 reaches an angle 0 as a result of backward rotation of the knee support 260 (the center of the arc of the knee support 260 is at an angle of −71°, the front first sensor 236 detects the indicator 265 to identify the reference position (see FIG. 9).

The rear indicator 265 is used to detect malfunction that the knee holder 250 moves out of the normal range of rotation and reaches the limit position, so that the rotational driving unit 240 can immediately stop the rotation of the knee holder 250.

When the reference position is detected, the knee support 260 is rotated forward by a predetermined amount of driving force to move from the reference position to a neutral position. In the neutral position, the center of the arc is at 0°. (see FIG. 10).

In the standby state, exercise stage and the number of rotations per set can be set. There are stages 1 to 8. The number of rotations per set in stage 1 has to be set 30 times. The number of rotations per set in stage 2 can be selectively set 30 times or 40 times. In the remaining stages from stage 3, the number of rotations per set can be selected from 30, 40 or 50 times.

The rotational driving unit 240 increases the maximum forward rotation angle of the knee holder 250 in response to the stage-up of the exercises. For example, the knee support 260 has maximum forward rotation angles, such as 65° in stage 1, 75° in stage 2, 85° in stage 3, 95° in stage 4, 105° in stage 5, 115° in stage 6, 125° in stage 7 and 135° in stage 8.

The exercise intensity is not selected by the user. Rather, exercise intensity (rotation range) for respective stages to yield optimum correction effects without applying excessive load to the body, are previously examined through clinical tests, and the optimum exercise intensity is selected to maximize the correction effects.

The controller stores the exercise history of the user in the memory, and searches for a rotation condition corresponding to the exercise history of the user stored in the memory in order to control the rotational driving unit 240 according to the searched rotation condition.

Describing in greater detail, the controller restricts exercise stages according to the exercise history of the user so that the user can make a selection out of the restricted exercise stages. The exercise history includes data of the exercise stages of the user and the number of remaining sets necessary for a stage-up. The stage-up is impossible until the operation of the knee holder 250 is accomplished by a predetermined number of sets in the present stage.

In order to stage-up, that is, proceed to the next step, respective stages need pre-determined number of exercise sets to be completed, for example, 10 sets (in case of 30 rotations per set) in stage 1, 20 sets (in case of 40 rotations per set) in stage 2, 30 sets (in case of 50 rotations per set) in stage 3, 40 sets (in case of 50 rotations per set) in stage 4, 50 sets (in case of 50 rotations per set) in stage 5, 60 sets (in case of 50 rotations per set) in stage 6, and 70 sets (in case of 50 rotations per set) in stage 7. However, even if the user exercises four (4) or more sets one day, up to three (3) sets are reduced per day.

If the user has not exercised for two (2) consecutive days in a specific stage, the amount of the remaining sets in this stage is added as follows:

D=1: No set added,

2≦D≦5: Sets added by a number corresponding to D×1,

6≦D≦10: Sets added by a number corresponding to D×2, and

11≦D: Sets added by a number corresponding to D×3,

wherein D represents days for which the user did not exercise.

Once this stage has the maximum set number by the addition of the sets, the exercise stage falls down to the lower stage.

The correction device can have “HISTORY INITIALIZATION” button to initialize the exercise history to a stage 1 with thirty remaining sets.

In the standby state, the user wear the knee protector 271 on the knee, and then place the knee on the knee support 260, and the foot on the foot support.

When “START” button of the remote controller is pressed, the knee support is rotated to the starting position, in which the center of the arc is at −58° (see FIG. 11).

When the knee support 260 is at the starting position, the user connects the knee protector 271 to the knee support 260. As the knee is fixed when the knee support 260 is at −58 rather than at the neutral position, it is possible to enlarge the forward rotation angle of the knee support 260, i.e., the outward rotation angle of the knee.

After the buckle 277a of the connector belt 277 is fastened with the buckle 257a of the connector belt 257, the user presses “START” button again.

Then, the user rotates the foot outward.

When the foot support 341 is rotated forward to a predetermined angle (e.g., 15° or more) and the rotation block 343 remains in contact with the switch of the third sensor 349 for a predetermined time period (e.g., 5 seconds), the forward rotation of the knee holder 250 is started.

If the foot support 341 is rotated forward less than the predetermined angle (e.g., 15°) or the rotation block 343 does not remain in contact with the switch of the third sensor 349 for the predetermined time period (e.g., 5 seconds), an alarm sound is generated.

FIG. 12 illustrates the rotation angle of the knee support 260 that has completed the first forward rotation, in which the center of the arc of the knee support is, for example, at −14°.

When the foot support is rotated forward after the completion of the first forward rotation and is maintained in this position for three (3) seconds, the knee support 260 starts the first backward rotation. If seven (7) or more seconds passed without the forward rotation of the foot support 341, an alarm sound will be generated. When the foot support 341 is rotated for the predetermined time period, the alarm sound will stop.

FIG. 13 illustrates the rotation angle of the knee support 260 that has completed the first backward rotation, in which the center of the arc of the knee support is, for example, at −52°.

Since the knee joint and the ankle joint are joints that cannot be rotated, the present invention forces the user to rotate his foot outward to obtain the outward rotation of the hip joint, thereby enhancing the correction effects for bowleg deformity. That is, the forward or backward rotation of the knee holder 250 is set to start only after the forward rotation of the foot support 341 to force the user to rotate the foot support 341 forward every time. Accordingly, it is possible to enhance the correction effect by stretching the ligament in the front part of the hip joint through the outward rotation of the knee as well as strengthening the abductor of the hip joint through the outward rotation of the foot.

Also, the abductor of the hip joint can be effectively strengthened by forcing the foot support 341 to keep its position after the forward rotation thereof, for a pre-determined time period. After the forward rotation of the knee holder 250, the knee holder 250 keep its position for at least the time period (e.g., 3 seconds) taken for the outward rotation of the foot support 341, thereby effectively stretching the ligament in the front part of the hip joint, which consequently enhances the correction effects. Even if the user starts the forward rotation of the foot support 341 immediately after the forward rotation of the knee support 260 is completed, backward rotation cannot start for a specific time period necessary for starting the backward rotation of the knee support 26. Thus, for at least this time period, the knee support 260 remains in the position where its forward rotation is completed.

While the forward rotation of the foot support 341 can be skipped while the knee holder 250 is rotating, it is advantageous in terms of effectiveness to continue the forward rotation of the foot support 341 if possible.

In addition, during the forward rotation of the foot supports, it is preferable to force the knees toward each other.

Through the repetition of these procedures, the forward and backward rotations of the knee support 260 are performed by predetermined numbers. For storing the correction device after the exercise, the user is recommended to press “READY” button to rotate the knee support 260 to the neutral position.

The rotation ranges of the knee holder 250 will now be described with reference to FIG. 14. In an early period, the zone where the knee holder 250 rotates forward/backward, gradually moves in a forward direction. Finally, in the last period, the zone gradually moves in a backward direction.

The exercise intensity is gradually raised, so that the user can warm up in the early period and do the main exercise in the middle period. In the last period, the exercise intensity is gradually lowered, so that the user can cool down. Accordingly, maximum treatment effects can be obtained without applying excessive load to the body.

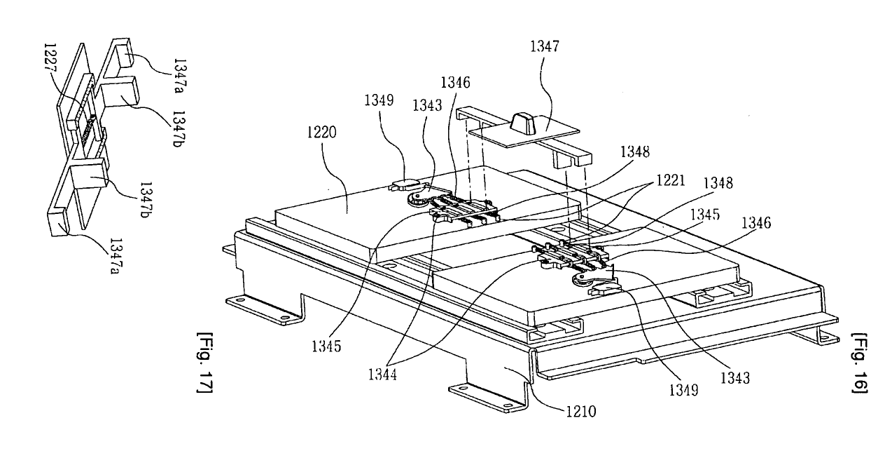

FIG. 15 is a perspective view schematically illustrating the external appearance of a correction device according to another embodiment of the present invention.

As shown in FIG. 15, the correction device is similar to the assembly 300 separated from the correction device shown in FIG. 1. The correction device shown in FIG. 15 strengthens the abductor of the hip joint through the outward rotation of feet, thereby correcting bowleg.

The correction device includes a housing 1351, foot supports 1341, a display 1353, a handle 1355 and a controller (not shown).

The foot supports 1341 receive and support feet, and are designed to be rotatable forward or backward in the direction of the outward or inward rotation of the feet.

The correction device has the handle 1355 that a standing user can seize. The handle 1355 is arranged in such a position that the user can seize it with hands when he/she is standing with the feet seated in the foot supports 1341. The handle 1355 can be designed to be detachable from and attachable to the housing 1351.

In addition, the correction device shown in FIG. 16 can be used when the user is in a lying position or a seated position.

FIG. 16 is a perspective view illustrating the internal structure of the correction device shown in FIG. 15, and FIG. 17 is a bottom perspective view of a switch locker 1347 shown in FIG. 16.

The correction device includes third resisting means 1346, a fourth sensor 1349 and fourth resisting means 1227 in addition to the housing 1351, the foot supports 1341, the display 1353 and the handle 1355.

The third resisting means 1346 act similar to the second resisting means illustrated with reference to FIG. 7, the fourth sensor 1349 act similar to the third sensor 349 shown in FIG. 7, and the fourth resisting means 1227 act similar to the first resisting means 227 shown in FIG. 3.

A pair of seats 1220 is mounted on a base 1210 and can move to the right and left. The foot supports 1341 are also mounted on respective seats 122 and can rotate forward and backward. Therefore, the foot supports 1341 can freely-move laterally within a fourth preset range.

Rotation blocks 1343 rotate along with the foot support 1341.

Through the contact with the rotation blocks 1343, the fourth sensor 1349 detects whether or not the foot supports 1341 keep forward rotation at a predetermined angle or more for at least a predetermined time, and outputs the number of the forward rotation of the foot supports 1341 on the display 1353. The angle of the forward rotation of the foot supports 1341 detected by the fourth sensor 1349 can be set adjustable.

The third resisting means 1346 are connected to one end of the rotation blocks 1343 and link blocks 1345.

Another resisting means 1348 are connected to the other end of fixing parts 1221 which are formed on the seats 1220, and the other end of the link blocks 1345.

The switch locker 1347 has insert protrusions 1347a and fitting protrusions 1347b which protrude downward. Each insert protrusions 1347a is inserted into the groove of a corresponding link block 1345, and each of the fitting protrusions 1347b is caught by a respective substrate 1344 which is fixed to a respective seat 1220. Accordingly, when the insert protrusion 1347a of the switch locker 1347 is inserted into the groove of the link block 1345, this link block 1345 is fixed in position with respect to the substrate 1344 and the seat 1220.

The fourth resisting means 1227 are provided to the switch locker 1347. The fourth resisting means 1227 resist against movement of the link blocks 1345, of the substrates 1344, of the seats 1220, of the rotation blocks 1343 and of the foot supports 1341 to the right and left toward each other. That is, the fourth resisting means 1227 resist against the foot supports 1341 moving to the right and left toward each other. The resisting force of the fourth resisting means 1227 can be adjusted.

The usage of the correction device will now be described.

First, the user moves the switch locker 1347 to a desired position so that the insert protrusions 1347a of the switch locker 1347 are inserted into the grooves of corresponding link blocks 1345.

When the user moves the feet toward each other, the foot supports 1341, the rotation blocks 1343, the seats 1220, the substrates 1344 and the link blocks 1345 also move toward each other. Accordingly, the insert protrusions 1347a also move to the right and left toward each other and the fourth resisting means 1227 resist against such movement.

When the user rotates the feet outward in this state, the foot supports 1341 and the rotation blocks 1343 rotate forward and the third resisting means 1346 resist against the rotation. When the user rotates the feet outward, the rotation blocks 1343 pull the third resisting means 1346 but the fitting protrusions 1347b of the switch locker 1347 caught by the substrates 1344, restrain the movement of the link blocks 1345, so that the resisting force of the third resisting means 1346 is directly applied to the rotation blocks 1343.

1.-23. (canceled)

24. A device for correcting bowleg, comprising:
a pair of foot supports for receiving and supporting feet, the foot supports capable of performing
forward and backward rotations in directions of rotating the feet outward and inward, respectively; and
a third resisting means for resisting against the forward rotation of the foot supports.

25. The device according to claim 24, further comprising a sensor for detecting the forward rotation of the foot supports at least a predetermined angle.

26. The device according to claim 25, wherein the sensor detects whether the forward rotation of the foot supports at least the predetermined angle is maintained for at least a predetermined time.

27. The device according to claim 25, wherein the predetermined angle of the forward rotation of the foot supports detected by the sensor is adjustable.

28. The device according to claim 24, wherein the third resisting means has an adjustable resisting force.

29. The device according to claim 24, wherein the foot supports are laterally freely-movable within a fourth preset range.

30. The device according to claim 29, further comprising a fourth resisting means for resisting against the foot supports moving toward each other within the fourth preset range.

31. (canceled)

32. The device according to claim 24, wherein each of the foot supports includes a footing cushion,
wherein the footing cushion comprises inner and outer plies, and
wherein the inner ply directly contacts the foot and is made of a softer material than the outer ply.

33. The device according to claim 24, further comprising a handle, wherein the handle is arranged in such a position that a user standing with the feet received and supported on the foot supports can seize the handle.
